(19) United States
(12) Patent Application Publication
(10) Pub. No.: US 2016/0287554 A1
(43) Pub. Date: Oct. 6, 2016

(54) Small Molecule Metal-Activated Protein Inhibition

(71) Applicants: Wayne Charles Guida, St. Petersburg Beach, FL (US); Robert Pleasants Sparks, Tampa, FL (US); Kenyon Gregory Daniel, Wesley Chapel, FL (US); Rainer Scott Metcalf, Palm Harbor, FL (US); Sreya Mukherjee, Tampa, FL (US); Wesley Harrell Brooks, Safety Harbor, FL (US)

(72) Inventors: Wayne Charles Guida, St. Petersburg Beach, FL (US); Robert Pleasants Sparks, Tampa, FL (US); Kenyon Gregory Daniel, Wesley Chapel, FL (US); Rainer Scott Metcalf, Palm Harbor, FL (US); Sreya Mukherjee, Tampa, FL (US); Wesley Harrell Brooks, Safety Harbor, FL (US)

(73) Assignee: University of South Florida, Tampa, FL (US)

(21) Appl. No.: 15/088,785

(22) Filed: Apr. 1, 2016

Related U.S. Application Data
(60) Provisional application No. 62/142,623, filed on Apr. 3, 2015.

(51) Int. Cl. A61K 31/343; A61K 31/27; G06F 19/16; A61K 31/366; A61K 31/353; A61K 31/50; A61K 9/00; A61K 31/192

(52) U.S. Cl. CPC A61K 31/343; A61K 9/0053; A61K 9/0019; A61K 31/27; A61K 31/192; A61K 31/366; A61K 31/353; A61K 31/50; G06F 19/16; C40B 30/02

(57) ABSTRACT

A cancer treatment comprises the administration of a pro-drug compound identified by an in silico candidate identification screening. The pro-drug candidates are selected from a data base and calculations are carried out on the association of the pro-drug candidates to form a complex with a metal ion and a proteasome active site. The pro-drug inhibits the active site of the proteasome in the presence of the metal ion and has little or no effect in the absence of the metal ion. The pro-drug can be: 3,4-dihydroxybenzoic acid; galloflavin; 2-{[(carbamoylsulfanyl)acetyl]amino}benzoic acid; 6,7-Dihydroxycoumaranone; 3,6-bis(hydroxymethyl)pyridazin-4(1H)-one; and 4′,5,7-trihydroxyisoflavone, for binding with copper ion and proteasome.

### CROSS-REFERENCE TO A RELATED APPLICATION

This application claims the benefit of U.S. Provisional Application Ser. No. 62/142,623, filed Apr. 3, 2015, the disclosure of which is hereby incorporated by reference in its entirety, including all figures, tables and drawings.

### BACKGROUND OF INVENTION

Cancer therapy can be as debilitating as the disease to a patient as current treatments are often accompanied by severe toxicities. These toxicities prompt continuing investigation into new therapies with reduced or, preferentially, no toxic effects. While treating cancer cells without toxicity to normal cells is the goal of drug discovery, the task itself has met with limited success due to the difficulty of distinguishing cancer cells from normal cells. One direction for the desired differentiation is to focus on the elevated levels of copper in almost all types of cancers.

Copper, which has the ability to adopt both oxidized (Cu²⁺) and reduced (Cul₊) states, is an essential trace element for various metabolic processes in living organisms. There are several enzymes that use copper for processes necessary for carcinogenesis such as extracellular matrix degradation, endothelial cell proliferation, and migration mediated by integrins. Due to its role in important physiologic processes, including metabolism, the concentration of copper in organisms is tightly regulated. Copper is an element that plays an essential role in tumor development, angiogenesis, and metastasis. Experimental evidence exists that shows tumor tissues possess both elevated copper and altered copper/zinc ratios in a stage dependent manner across multiple types of carcinomas. Elevated serum copper levels in cancer patients have been reported in a wide variety of tumors in the following tissues: breast, cervical, ovarian, lung, prostate, and stomach.

For example, many investigators have shown that growth inhibitory effects, both in vitro and in vivo are specific to cancer cells for thymoquinone (TQ), a known anti-inflammatory, antioxidant and anti-neoplastic compound. Although a known antioxidant at low concentrations, a pro-oxidant effect has been demonstrated for TQ. TQ's proposed activity is attributed to it causing DNA breakage in the absence of added copper ions in lymphocytes presumably through mobilization of endogenous copper ions where redox cycling of copper leads to the generation of reactive oxygen species to serve as the proximal DNA cleaving agent.

Inhibition of proteasomes, cellular complexes that break down proteins, is an emerging strategy for anti-cancer therapy. Degradation of cellular proteins is a highly complex and regulated process central to the regulation of cellular function and maintaining homeostasis. The ubiquitin proteasome pathway (UPP) is the major pathway for intracellular protein degradation, including protein degradation during processes including apoptosis. Defects within this pathway are associated with a number of diseases, including cancer.

Studies show that in cellulo assembled copper-activated proteasome inhibitors have apoptosis-inducing effects on a wide array of solid tumors and no measurable effect on normal cells. Yet, the field of copper-activated proteasome inhibitors has stalled due to lack of therapeutically suitable compounds. Only a very small number of organic scaffolds have been studied with respect to complexation with copper for proteasome inhibition in cancer cells, including: pyrrolidine dithiocarbamate; 8-hydroxyquinoline (8-HQ); clioquinol (CQ); and disulfiram. Prior studies have shown that these compounds have differential effects in immortalized, pre-malignant, and malignant breast cancer cells.

To such ends, compounds where in cellulo endogenous tumor copper activates the compound for inhibiting proteasome within cancer cells is desirable. Thereby, a selective induction of apoptosis in tumor cells due to the proteasome inhibition can be carried out by a pro-drug compound that is selectively activated in tumor cells while remaining inactive toward proteasome inhibition in normal cells and greatly reducing adverse effects on patients.

### BRIEF SUMMARY

Embodiments of the invention are directed to a cancer treatment formulation that has a dosage form for administration of at least one pro-drug compound that will combine with at least one metal ion that is inherently is at elevated levels in cancer cells. The pro-drug compound is selected by in silico screening for association with the selected metal ion and a proteasome for indication that the association with the proteasome occurs only, or to a significantly greater extent, in the presence of the metal ion. The metal ion can be an ion of copper, zinc, nickel, or iron. Exemplary pro-drug compounds include: 3,4-dihydroxybenzoic acid; galloflavin; 2-[(carbamoylsulfanyl)acetyl]amino benzoic acid; 6,7-Dihydroxycoumaranone; 3,6-bis(hydroxymethyl)pyridazin-4(1H)-one; and 4′,5,7-trihydroxyisoflavone.

In an embodiment of the invention, a computer program includes code for determining pro-drug candidates for proteasome inhibition, where in silico modeled of a candidate pro-drug compound with a metal ion and a proteasome active site indicates candidates when the calculated structure indicates that the pro-drug compound complex with the metal ion associates with a threonine-1 residue of the proteasome active site.

In another embodiment of the invention, a diagnostic or theranostic agent, is a formulation of a pro-drug compound that promotes apoptotic turnover by formation of complexes with metal ions that associate with proteasome active centers to induce elevated populations of apoptotic biomarkers in a patient's blood upon delivery of the formulation.

### BRIEF DESCRIPTION OF DRAWINGS

FIG. 1 shows the structure of disuliram, a known proteasome inhibitor, and six pro-drug compounds, according to an embodiment of the invention, that were identified by the computational method, according to an embodiment of the invention.

FIG. 2 shows an illustration of a complex of the pro-drug 16631 with copper ion docked in the proteasome, to inhibit the proteasome, according to an embodiment of the invention.

FIG. 3 shows an illustration of a complex of the pro-drug 107022 with copper ion docked in the proteasome, to inhibit the proteasome, according to an embodiment of the invention.

FIG. 4 shows an illustration of a complex of the pro-drug 13345 with copper ion docked in the proteasome, to inhibit the proteasome, according to an embodiment of the invention.

FIG. 5 shows an illustration of a complex of the pro-drug 37408 with copper ion docked in the proteasome, to inhibit the proteasome, according to an embodiment of the invention.

FIG. 6 shows an illustration of a complex of the pro-drug 166900 with copper ion docked in the proteasome, to inhibit the proteasome, according to an embodiment of the invention.

FIG. 7 shows an illustration of a complex of the pro-drug 36586 with copper ion docked in the proteasome, to inhibit the proteasome, according to an embodiment of the invention.

FIG. 8 is a bar graph of pro-drug compound NSC 37408 activity with the proteasome in the absence of copper ion at various concentrations from the data of Table 3.

FIG. 9A is a bar graph of the activity of 20 μM pro-drug compound NSC 37408 in the presence of various concentrations of copper ion.

FIG. 9B is a bar graph of the activity of 2 μM pro-drug compound NSC 37408 in the presence of various concentrations of copper ion.

FIG. 10 is a bar graph of the activity of the pro-drug compound NSC 37408 at various concentrations in the absence of copper ion from the data of Table 6.

FIG. 11 is a plot of the dose response of copper chloride on the % activity of the proteasome where an inhibitory effect displays an EC50 value of 0.686 μM.

FIG. 12 is a plot of the dose response curve for NSC 37048 with copper at 1 μM concentration and the comparative inhibition at 0 μM copper concentration.

FIG. 13 shows composite plots for the dose response for NSC 34708 with copper alone (circles), NSC 34708 alone (squares), and NSC 34708 with copper (triangles), where a dramatic improvement of percent inhibition is observed for the compound in the presence of sub-micromolar copper, where that concentration of copper absent the pro-drug exhibits no significant inhibitory activity for experiments in triplicate with a relative error of less than 10% for all experiments.

### DETAILED DISCLOSURE

Embodiments of the invention are directed to cancer treatment formulations and methods that exploit a tumor's metal ion loading to physiologically differentiate the effect in cancer cells and normal cells of pro-drug compounds that are benign in the absence of the elevated cellular metal ions of malignant cells. These pro-drug compounds mobilize endogenous tumor metal ions, such as copper ions, resulting in in cellulo metal complexes or other metal ion mediated derivatives that are active drugs for inhibiting the proteasome within cancer cells. In this manner, selective induction of apoptosis in tumor cells occurs. Although embodiments of the invention will be described herein as the pro-drug mediated by copper ions, the invention is not so limited, and other metal ions, for example, but not limited to, zinc, nickel, or iron, have the capability to display similar or greater advantages in therapies that provide selective targeting of tumor cells.

Pro-drugs, according to an embodiment of the invention, are mono-dentate complexing agents, bi-dentate or other chelating agents that bind to metal ions in patients adversely affected by tumors with elevated metal ion levels, such as hemochromatosis, in which excess iron can cause organ toxicity. In another embodiment of the invention, the pro-drug compounds can be employed to effectively remove radioactive metal ions from patients or objects that have been exposed to radioactive materials.

In other embodiments of the invention, these pro-drug compounds can be employed as diagnostic and/or prognostic agents that are safe and efficient. For example, in difficult and aggressive cancers, such as pancreatic cancer, where late detection of the cancer may preclude effective countermeasures, the compounds can be used upon suspicion for the early detection of cancer without adversely affecting the patient. The compound, when administered as a diagnostic or theranostic agent, induces increased apoptotic biomarkers in the subject's blood, above the normal background of apoptotic turnover, due to the presence of tumor cells somewhere in the body. In another embodiment of the invention, an elevation in a patient's blood serum copper level is suspicious of a hidden cancer, and the pro-drug compound is administered as a theranostic where subsequent changes in the serum copper level are monitored for additional release of copper due to induced tumor cell death. The compound, when used as a theranostic, would provide data for the presence of cancer and provide support for administering the pro-drug compound or a similar pro-drug compound according to an embodiment of the invention, in sufficient amounts to reduce or vanquish the tumor cells. Prognostic follow-up monitoring of the therapeutic effect could entail additional administrations of the pro-drug compound and measurement of the apoptotic activity and copper levels to determine if the activity has returned to a base line, which indicates clearance or at least remission of the cancer. Hence, in an embodiment of the invention, the pro-drug compounds are used to detect the presence of cancer by monitoring apoptotic biomarkers and/or copper levels compared to base line levels preceding the pro-drug compound's administration.

The pro-drug compounds, according to embodiments of the invention, can function in synergy with one or more other pro-drug compounds or other drugs according to embodiments of the invention, or with other cancer therapies. Individually, different drugs can have different rates of activity due to differences in absorption, distribution, metabolism and elimination that can be exploited for effective therapy. By developing and optimizing combinations of these compounds, there could be benefits by the allowance of lower doses of each compound and/or extension of or reduction in the length of time that the tumor cells are exposed to the therapeutic effects.

As a dosage formulation, according to an embodiment of the invention, one or more of these pro-drug compounds are combined with other components, such as buffering agents, transporters, salts, fillers, and/or encapsulation. These components can be combined with the pro-drug compound(s), for a desired mode of delivery, or for an improvement of therapeutic effectiveness of these pro-drug compounds, by improving absorption, distribution, metabolism, and excretion (ADME) characteristics and/or improving the timing of therapeutics, such as time release modifications. The pro-drug compound can be administered intravenously, orally, rectally, sublingually, sublabially, epidurally, intracerebrally, intracerebroventrically, topically, nasally, intervitrally, subcutaneously, transdermally, by inhalation, or in any other manner of administration.

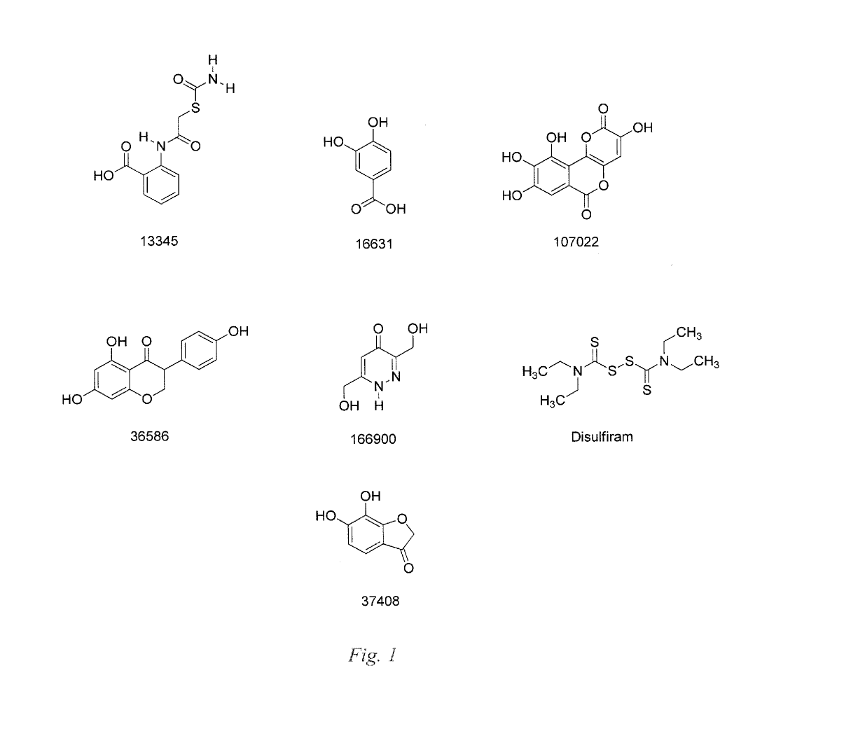

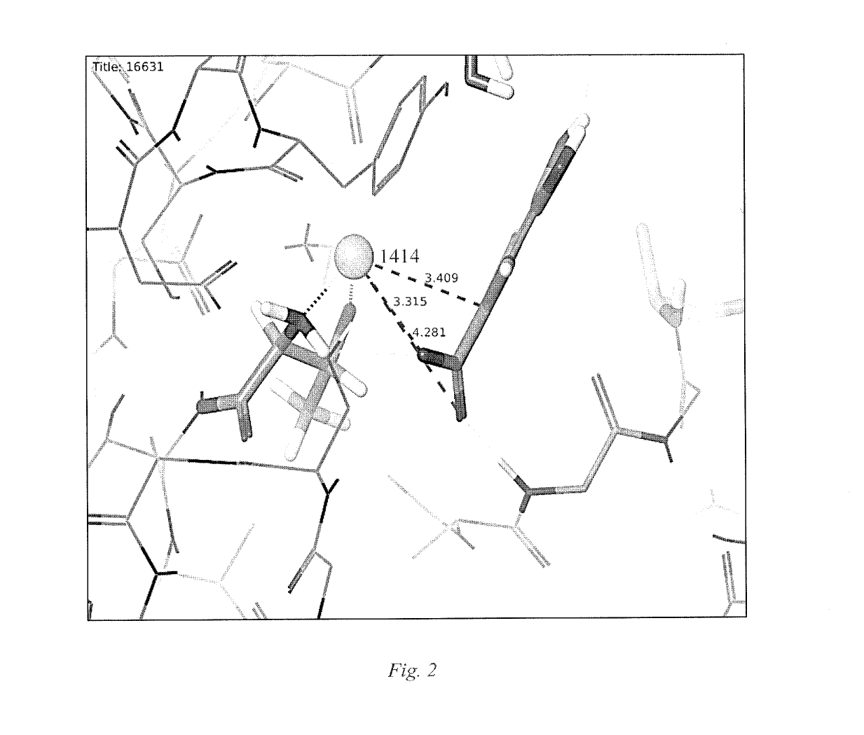

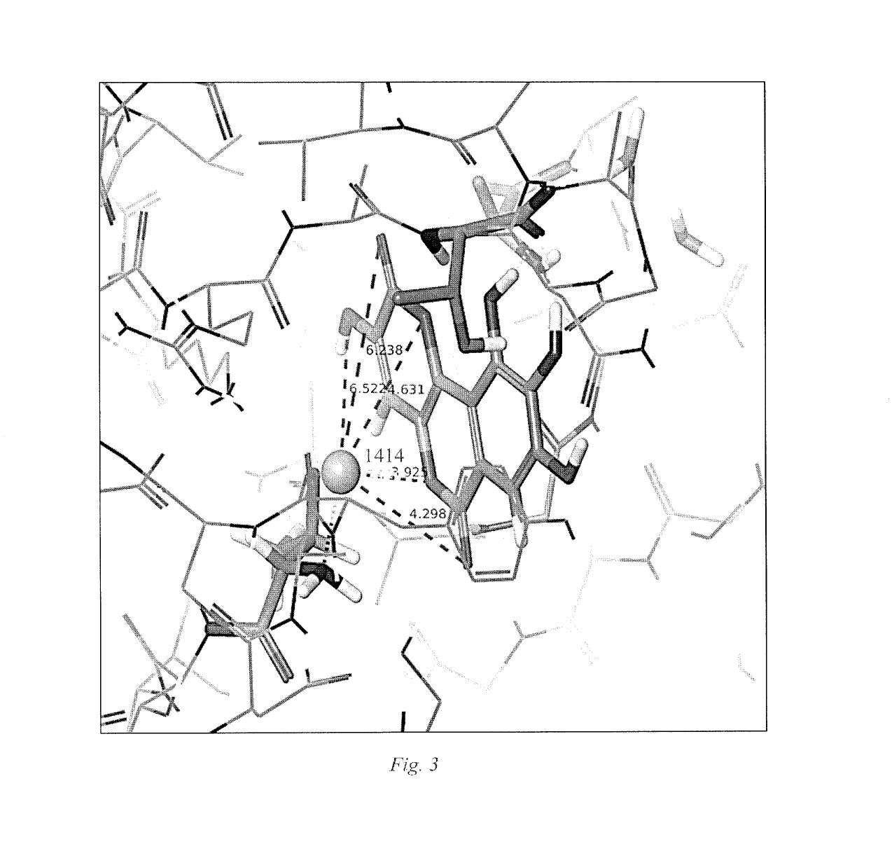

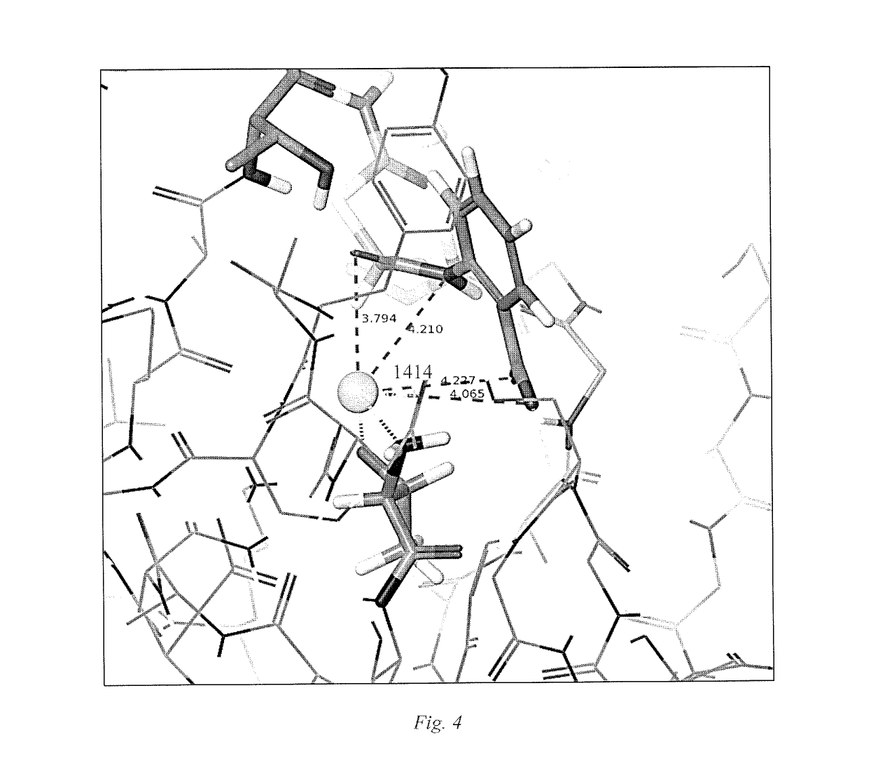

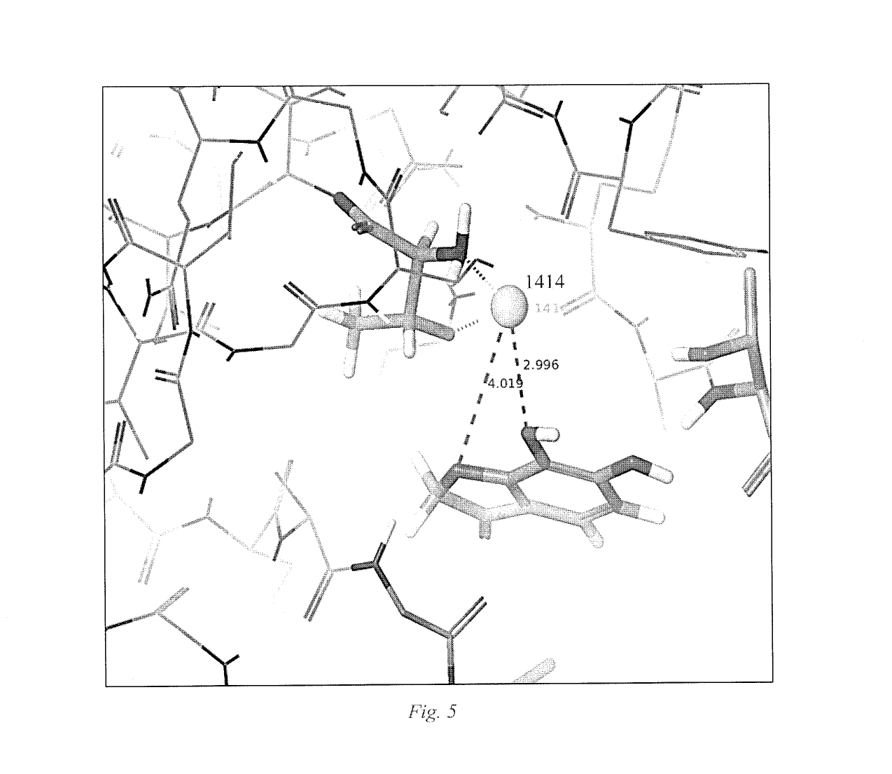

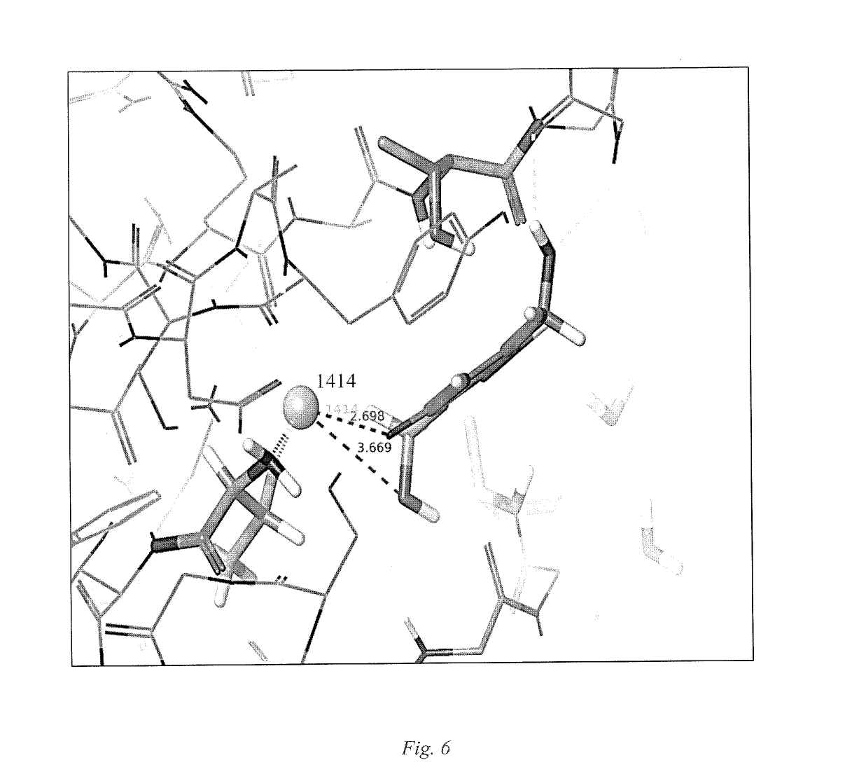

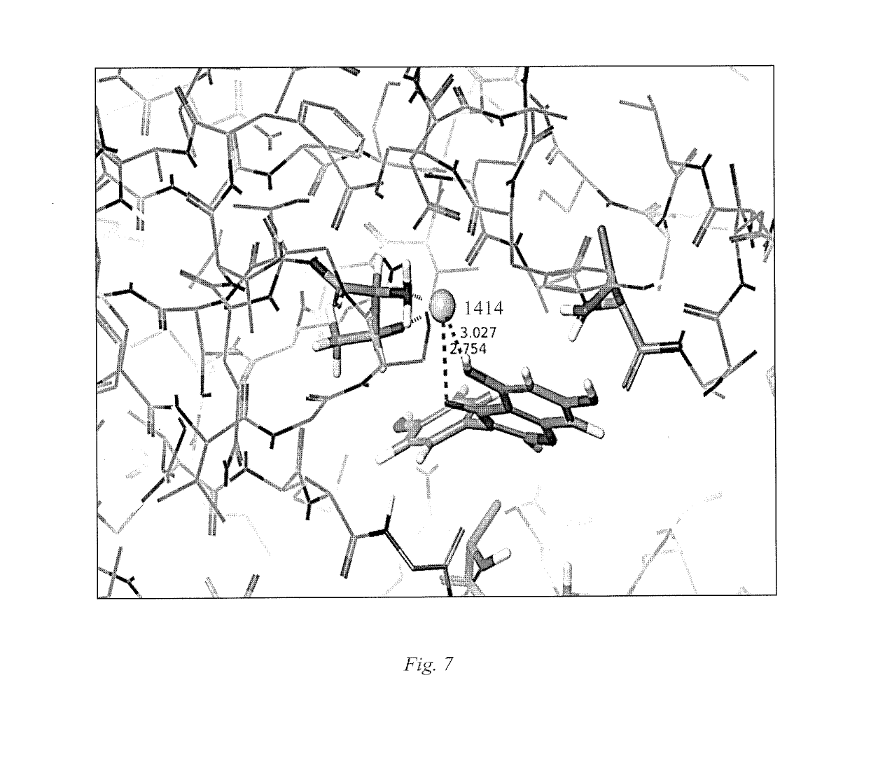

Pro-drug compounds, according to embodiments of the invention, include: 3,4-dihydroxybenzoic acid; galloflavin; 2-{[(carbamoylsulfanyl)acetyl]amino}benzoic acid; 6,7-Dihydroxycoumaranone; 3,6-bis(hydroxymethyl)pyridazin-4(1H)-one; and 4′,5,7-trihydroxyisoflavone, as shown in FIG. 1. These compounds, and analogs thereof, can act individually or can be used in combination. These compounds, and similar compounds, are capable of binding copper ions and facilitating inhibition of the proteasome. These compounds, and similar compounds, are safe and efficacious theranostics and/or drugs to detect and/or treat cancers. These compounds are from the NCI Diversity Set 3. Preliminary identification of these compounds as potential Cu-complexes with the proteasome was conducted using an in silico screening method, according to an embodiment of the invention, for example, with graphic representations of the calculated docking of the pro-drugs of FIG. 1 with copper ion and the proteasome, as shown in FIGS. 2-7. The modeling method indicates the potential of candidates as the pro-drugs, which are tested experimentally to confirm their efficiency using an in vitro experimental assay that tests for inhibition of the chymotrypsin-like activity of the 20S proteasome.

The in silico screening method models the interactions of the compounds with copper ions and the proteasome active site. The computer program allows the copper ion complexed with small organic molecules to be modeled in mono-dentate and bi-dentate forms and docked with the proteasome to estimate a potential location to place the copper in the active site. After placement of the copper, the docking region in the active site with the placed copper was constructed and the small ligands were docked to this modified active site. The placement of copper was subsequently modified using rigorous quantum mechanical procedures based on the active site residue, threonine-1, which is well-established as being one of the most important residues in the active site for the proteasome activity. The model used both free and zero-order bonds to copper, where the zero-order bonds are assigned based upon modifications for inclusion of ligands to the metal binding sites. Partial charges were inputted manually to the proteasome model based on the quantum mechanical calculations. Without the normal proteasome activity, abnormal proteins accumulate and lead to cell death. In an embodiment of the invention, this method of virtually modeling the pro-drug compound, its metal ion interactions, and the pro-drug compound metal ion complexes interactions with the proteasome, is used to rationally identify promising candidates for pro-drug compounds to be experimentally tested for confirmation of the activity of these compounds to inhibit proteasome selectively in cells with elevated metal ion levels.

### Methods and Materials

Compounds were selected from the NCI Diversity Set 3. Initially, 62 compounds from the NCI Diversity Set IV (˜1500 compounds) were selected by rational virtual screening and tested biochemically in one-dose experiments to determine the efficacy of the copper complex and its capacity to inhibit the proteasome. The six lead compounds, shown in FIG. 1, with their NSC numbers, are: 16631, 3,4-dihydroxybenzoic acid; 107022, galloflavin; 13345, 2-{[(carbamoylsulfanyl)acetyl]amino}benzoic acid; 37408, 6,7-dihydroxycoumaranone; 166900, 3,6-bis(hydroxymethyl)pyridazin-4(1H)-one; and 36586, 4′,5,7-trihydroxyisoflavone. Disuliram, a known proteasome inhibitor, is also displayed in FIG. 1. These six compounds were found to have superior inhibition against the proteasome.

Virtual screening was performed against the 20S proteasome model. Schrödinger's Maestro 9.3.5 was used as the primary graphical user interface for molecule structure preparation and Schrödinger applications were used for analysis. Quantum mechanical refinement of copper interactions with the THR1 in the active site using Q-site and Jaguar with B3LYP/LACVP*allowed for placement of copper and the assignment of partial charges on THR1 and the copper ion. The virtual screening method employed the modified yeast 20S proteasome crystal structure derived from PDB ID: 1IRU. Ligands from the NCI Diversity Set 3 were prepared with LigPrep 32 and metal binding sites were added for generation of appropriate ligand states to interact with the copper ion. The standard precision (SP) setting in GLIDE was used for docking to incorporate metal binding sites. Out of 1597 compounds, 62 were selected by the virtual screening method, which were then tested at 10 μM in the presence of 1 μM copper.

A Beckman Coulter Biomek FX^{P} Lab Automation Workstation was used for automating the assays. A Perkin Elmer EnVision 2102 multilabel plate reader was used to read the plates by fluorescence measurements. Assays were performed in 384 well black Nunc plates. Each compound (2 μl in DMSO), when tested without copper chloride was added to 28 μl of buffer (50 mM Tris at pH 7.6 and 37° C.), with 20 μM, 10 μl 20S proteasome and 10 μl of suc-leu-leu-val-tyr-AMC as the substrate (purchased from Boston Biochem), and the rate of substrate cleavage by 20S proteasome activity was determined. In the presence of copper, the buffer amount was decreased to 25 μl and 3 μl copper chloride was used for chelation with the compound for testing in the presence of copper. To allow chelation of copper to the compound, the plate was allowed to sit for 40 minutes with gentle shaking. The overall volume per well was held at 50 μL. The compounds and substrate were initially dissolved in 100% DMSO, but the final concentration of DMSO per well plate was reduced to less than 2% following subsequent dilutions. Plates were incubated at 37° C. for 2 hours. For dose response curves, the concentrations of copper chloride and a pro-drug compound, were varied to get optimal activity. The plates were read with 340 nm excitation and 460 nm emission using the plate reader. All liquid transfers to the plates were automated using the Biomek workstation.

Table 1 shows raw data from a first round of testing of 24 compounds identified by calculation. In table 1, the boxes in red (underlined) show activity with and without copper for the pro-drug compound NSC 37408, which shows better activity than does copper alone. Control tests included 2% dimethylsulfoxide (DMSO) instead of the pro-drug compound for full signal and was included for tests of only substrate and for copper controls, used to test inhibition of the proteasome by copper alone. The first four rows depict compounds without copper and the subsequent four rows depict the same compounds complexed with copper. Based on the data shown in Table 1, NSC 37408 showed better capacity to inhibit the proteasome than copper chloride alone to a large extent and was chosen as the lead for subsequent dose-response studies.

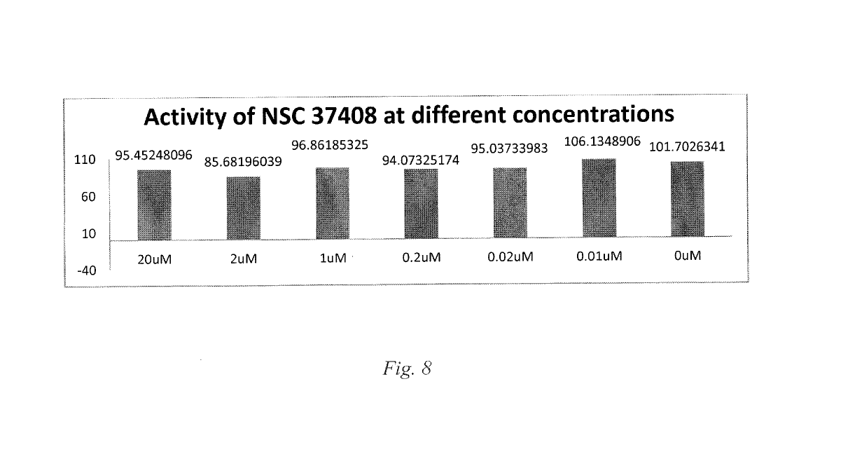

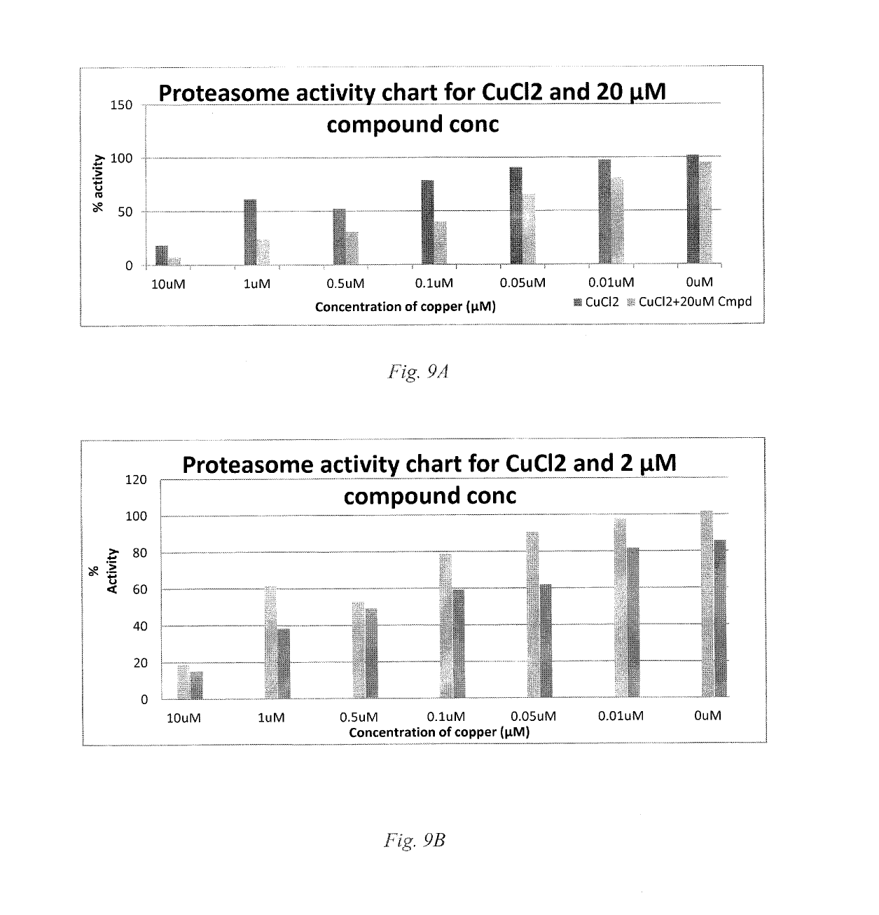

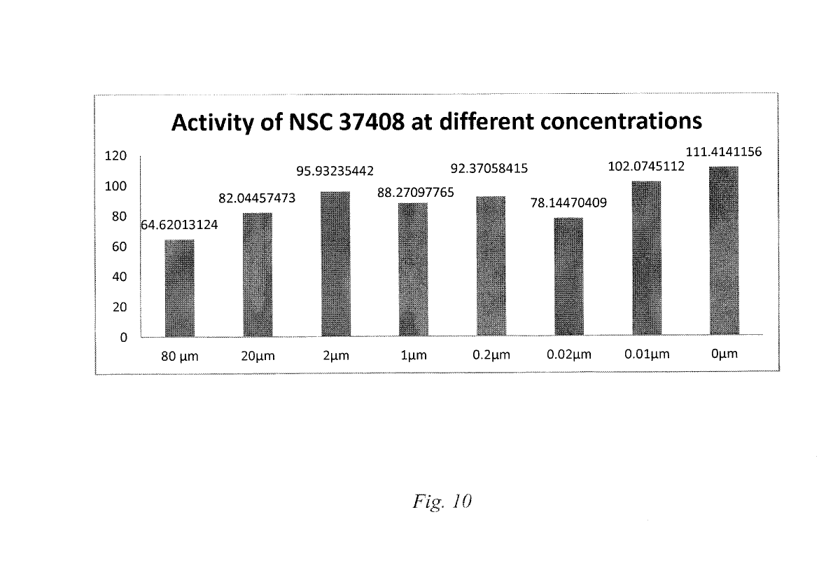

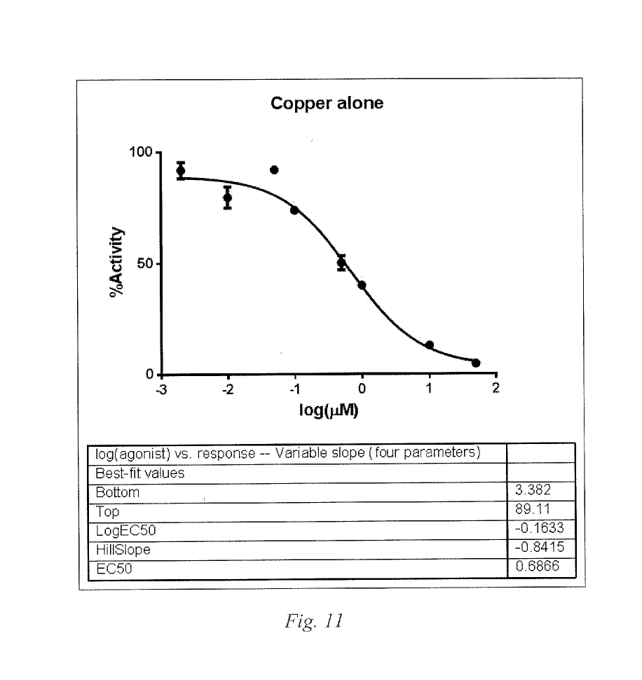

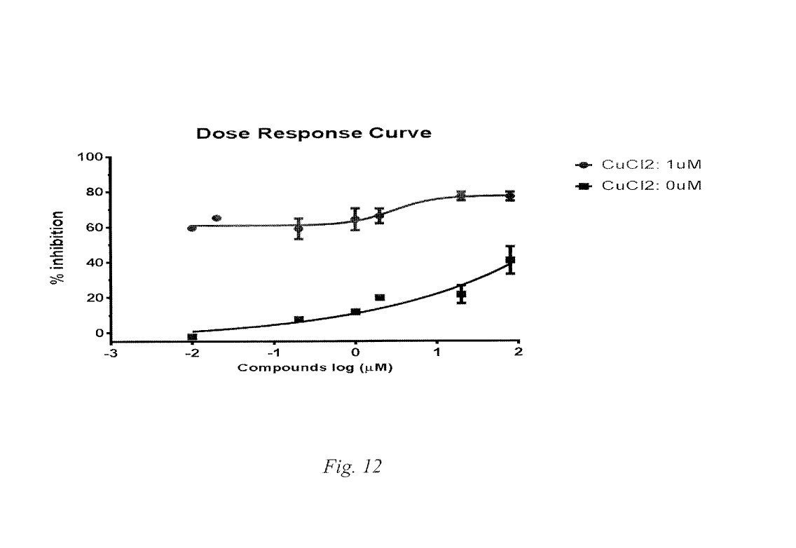

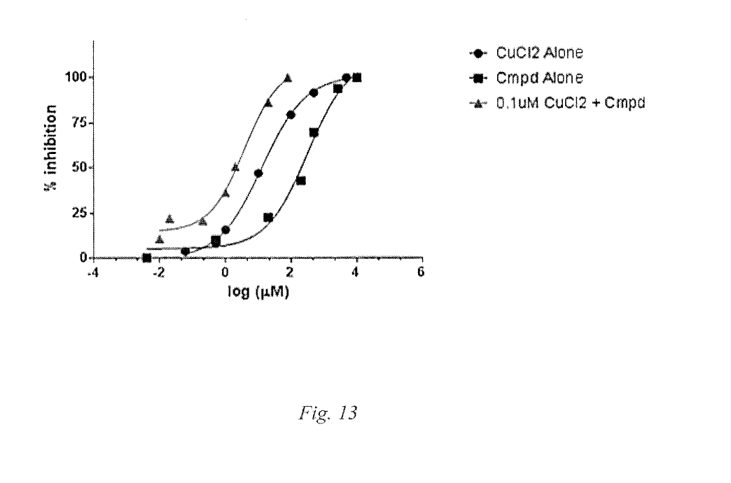

Table 2 gives dose response raw data for pro-drug compound NSC 37408, where columns 1 and 2 give controls. Subsequent columns display data for duplicate test conditions. Table 3 shows data for the conditions employed to establish a dose response curve for the pro-drug compound NSC 37408, tabulated for varied pro-drug compound NSC 37408 concentrations in the presence of different concentrations of copper. FIG. 8 gives % activities of the pro-drug NSC 37408 without copper chloride at various concentrations, while FIG. 9A and FIG. 9B give the % activities for two concentrations of the pro-drug NSC 37408 with copper chloride at various concentrations. Table 4 gives the % activities of the pro-drug compound NSC 37408 with copper chloride at various concentrations. Table 5 gives additional test results for determination of the dose response for pro-drug compound NSC 37408. Table 6 give the % activity data and Table 7 the % inhibition determined from the data of Table 5 for various concentrations of copper chloride and the pro-drug compound. Again, even with additional data, as indicated in FIG. 10, the pro-drug compound NSC 37408 displays little if any inhibition in the absence of copper ion. The copper ion does show a dose response absent the pro-drug compound, as indicated in FIG. 11, whereas the pro-drug compound requires the copper ion for significant inhibition, as indicated in FIG. 12. In a dose-response assay, NSC 37408 gave the best results with an IC₅₀ of 3 μM in presence of 100 nM copper, as indicated in FIG. 13.

Table 8 shows data for single dose testing of 38 compounds that includes the same controls as for the data of Table 1. This data identifies five additional potential pro-drug compounds and reconfirms the activity of the lead pro-drug compound NSC 37408. Table 9 give % inhibition calculated from the data of Table 8 where the first 4 rows indicate inhibition absent copper ion and the last four rows indicate inhibition with copper ion present. Table 10 give the % inhibition for pro-drug compound NSC 37408 and the additional five compounds of FIG. 1. All of which display superior abilities to inhibit the proteasome in the presence of copper ion in excess of the inhibition of copper ion to inhibit the proteasome. For comparison purposes, disulfiram, a popular metal chelating drug is included in Table 10 for comparison.

It should be understood that the examples and embodiments described herein are for illustrative purposes only and that various modifications or changes in light thereof will be suggested to persons skilled in the art and are to be included within the spirit and purview of this application.

TABLE 1
Compound screening

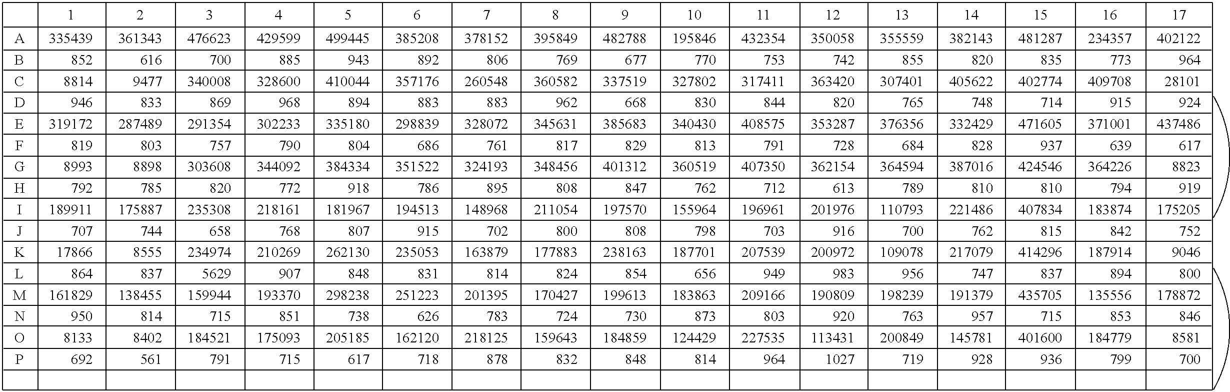

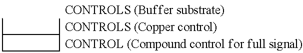

TABLE 2
Dose response raw data for NSC 37408.

| | 1 | 2 | 3 | 4 | 5 | 6 | 7 | 8 |
|---|---|---|---|---|---|---|---|---|
| A | 249696 | 214533 | 22033 | 21344 | 38142 | 37566 | 35679 | 39994 |
| B | 929 | 756 | 938 | 840 | 920 | 957 | 928 | 903 |
| C | 208783 | 187753 | 58865 | 53334 | 90856 | 79152 | 124736 | 91807 |
| D | 803 | 974 | 963 | 1087 | 1417 | 811 | 843 | 860 |
| E | 214370 | 190314 | 81560 | 58618 | 110509 | 103696 | 121388 | 117284 |
| F | 989 | 1027 | 982 | 1028 | 889 | 882 | 927 | 1023 |
| G | 895 | 1145 | 106928 | 69024 | 135375 | 120004 | 140419 | 128717 |
| H | 925 | 992 | 857 | 1050 | 999 | 1017 | 982 | 937 |
| I | 853 | 1205 | 118942 | 162067 | 132353 | 133185 | 142466 | 145237 |
| J | 944 | 946 | 1115 | 1157 | 1029 | 909 | 1054 | 1175 |
| K | 6462 | 5787 | 153591 | 189848 | 185503 | 161720 | 154979 | 159918 |
| L | 788 | 890 | 702 | 794 | 1019 | 1018 | 846 | 694 |
| M | 7871 | 7741 | 210478 | 192782 | 205279 | 158112 | 217397 | 191614 |
| N | 1008 | 860 | 857 | 982 | 873 | 902 | 1003 | 929 |
| O | 7943 | 5483 | 950 | 934 | 814 | 998 | 919 | 1044 |
| P | 987 | 864 | 930 | 923 | 840 | 896 | 829 | 976 |
| | | Buffer& | | | Compound | | | |
| | | substrate | | | full signal | | | |

| | 9 | 10 | 11 | 12 | 13 | 14 | 15 | 16 |
|---|---|---|---|---|---|---|---|---|
| A | 46592 | 43880 | 43253 | 42764 | 46652 | 48704 | 50106 | 40033 |
| B | 895 | 881 | 826 | 947 | 813 | 856 | 873 | 924 |
| C | 122862 | 118997 | 121236 | 95949 | 97842 | 120666 | 141117 | 122975 |
| D | 654 | 836 | 763 | 823 | 789 | 1089 | 990 | 931 |
| E | 137828 | 133359 | 110948 | 127698 | 118400 | 132897 | 109156 | 118862 |
| F | 1003 | 841 | 949 | 1034 | 914 | 1036 | 864 | 1206 |
| G | 157547 | 129395 | 141292 | 132314 | 155492 | 162517 | 176022 | 158525 |
| H | 852 | 951 | 1027 | 1108 | 1000 | 834 | 947 | 926 |
| I | 180896 | 176095 | 145829 | 119732 | 182439 | 180714 | 195308 | 187519 |
| J | 967 | 1090 | 720 | 919 | 963 | 947 | 884 | 880 |
| K | 199411 | 163000 | 186628 | 191566 | 192280 | 188076 | 243811 | 168613 |
| L | 818 | 791 | 957 | 905 | 825 | 976 | 973 | 989 |
| M | 221912 | 175720 | 202868 | 198698 | 236659 | 210191 | 225713 | 203051 |
| N | 888 | 958 | 886 | 880 | 920 | 989 | 854 | 890 |
| O | 952 | 1079 | 962 | 1019 | 1103 | 975 | 1072 | 993 |
| P | 916 | 840 | 862 | 933 | 875 | 1079 | 870 | 869 |

TABLE 3
Dose response data for NSC 37408 in the presence of different concentrations of copper.

| | | % Activity | | | | | | | |
|---|---|---|---|---|---|---|---|---|---|
| | | control | control | 20 uM | 20 uM | 2 uM | 2 uM | 1 uM | 1 uM |
| | | 1 | 2 | 3 | 4 | 5 | 6 | 7 | 8 |
| 10 uM | A | 119.0111 | 101.7766 | 7.426386 | 7.088686 | 15.32191 | 15.03959 | 14.11472 | 16.22963 |
| | B | | | | | | | | |
| 1 uM | C | 98.95839 | 88.65093 | 25.4789 | 22.76798 | 41.15869 | 35.42219 | 57.76433 | 41.6248 |
| | D | | | | | | | | |
| 0.5 uM | E | 101.6968 | 89.90616 | 36.60243 | 25.35784 | 50.79124 | 47.45197 | 56.12337 | 54.11187 |
| | F | | | | | | | | |
| 0.1 uM | G | −2.93401 | −2.81147 | 49.03608 | 30.45814 | 62.97884 | 55.44503 | 65.45106 | 59.71554 |
| | H | | | | | | | | |
| 0.05 uM | I | −2.95459 | −2.78207 | 54.92451 | 76.06142 | 61.49766 | 61.90545 | 66.45436 | 67.81251 |
| | J | | | | | | | | |
| 0.01 uM | K | −0.20545 | −0.53629 | 71.90707 | 89.67776 | 87.54813 | 75.89134 | 72.58737 | 75.00813 |
| | L | | | | | | | | |
| 0 uM | M | 0.485148 | 0.421431 | 99.78916 | 91.1158 | 97.24097 | 74.12295 | 103.1804 | 90.54333 |
| | N | | | | | | | | |
| | O | 0.520438 | −0.68529 | −2.90705 | −2.91489 | −2.97371 | −2.88352 | −2.92224 | −2.86098 |
| CuCl2 | P | | | | | | | | |

| | | % Activity | | | | | | | |
|---|---|---|---|---|---|---|---|---|---|
| | | 0.2 uM | 0.2 uM | 0.02 uM | 0.02 uM | 0.01 uM | 0.01 uM | 0 uM | 0 uM |
| | | 9 | 10 | 11 | 12 | 13 | 14 | 15 | 16 |
| 10 uM | A | 19.46352 | 18.13428 | 17.82697 | 17.5873 | 19.49293 | 20.49868 | 21.18584 | 16.24875 |
| | B | | | | | | | | |
| 1 uM | C | 56.84583 | 54.95147 | 56.04887 | 43.65492 | 44.58274 | 55.7695 | 65.79317 | 56.90121 |
| | D | | | | | | | | |
| 0.5 uM | E | 64.18113 | 61.99073 | 51.0064 | 59.2161 | 54.65886 | 61.76429 | 50.12809 | 54.8853 |
| | F | | | | | | | | |
| 0.1 uM | G | 73.84603 | 60.04785 | 65.87894 | 61.47855 | 72.83881 | 76.28198 | 82.9012 | 74.32538 |
| | H | | | | | | | | |
| 0.05 uM | I | 85.2901 | 82.93698 | 68.10267 | 55.31172 | 86.04637 | 85.2009 | 92.35387 | 88.53624 |
| | J | | | | | | | | |
| 0.01 uM | K | 94.36488 | 76.51871 | 88.09953 | 90.5198 | 90.86975 | 88.80924 | 116.1267 | 79.26982 |
| | L | | | | | | | | |
| 0 uM | M | 105.3933 | 82.75318 | 96.05926 | 94.01542 | 112.6213 | 99.64849 | 107.2563 | 96.14896 |
| | N | | | | | | | | |
| | O | −2.90607 | −2.84382 | −2.90117 | −2.87323 | −2.83206 | −2.8948 | −2.84725 | −2.88597 |
| CuCl2 | P | | | | | | | | |

TABLE 4
Average % Inhibition based on raw data.

| | % inhibition | | | | | | | |
|---|---|---|---|---|---|---|---|---|
| | | 20 uM | 2 uM | 1 uM | 0.2 uM | 0.02 uM | 0.01 uM | 0 uM |
| NSC 37408 | CuCl₂ | 3 | 4 | 5 | 6 | 7 | 8 | 9 |
| 10 uM | A | 92.74246 | 84.81925 | 84.82783 | 81.2011 | 82.29287 | 80.0042 | 81.28271 |
| | B | | | | | | | |
| 1 uM | C | 75.87656 | 61.70956 | 50.30543 | 44.10135 | 50.1481 | 49.82388 | 38.65281 |
| | D | | | | | | | |
| 0.5 uM | E | 69.01987 | 50.8784 | 44.88238 | 36.91407 | 44.88875 | 41.78842 | 47.49331 |
| | F | | | | | | | |
| 0.1 uM | G | 60.25289 | 40.78807 | 37.4167 | 33.05306 | 36.32125 | 25.43961 | 21.38671 |
| | H | | | | | | | |
| 0.05 uM | I | 34.50703 | 38.29844 | 32.86657 | 15.88646 | 38.29281 | 14.37637 | 9.554945 |
| | J | | | | | | | |
| 0.01 uM | K | 19.20759 | 18.28026 | 26.20225 | 14.5582 | 10.69033 | 10.1605 | 2.301738 |
| | L | | | | | | | |
| 0 uM | M | 4.547519 | 14.31804 | 3.138147 | 5.926748 | 4.96266 | −6.13489 | −1.70263 |

TABLE 5A
Depiction of dose response data.

| | 1 | 2 | 3 | 4 | 5 | 6 | 7 | 8 | 9 | 10 | 11 | 12 |
|---|---|---|---|---|---|---|---|---|---|---|---|---|
| A | 260141 | 238523 | 8891 | 8794 | 17411 | 15579 | 48961 | 42593 | 59866 | 50080 | 96716 | 82248 |
| B | 859 | 1058 | 990 | 950 | 984 | 752 | 1066 | 920 | 811 | 1083 | 954 | 874 |
| C | 209818 | 144565 | 10756 | 10315 | 17292 | 17243 | 48534 | 42417 | 61463 | 53091 | 98471 | 93582 |
| D | 890 | 1111 | 806 | 1019 | 990 | 775 | 1102 | 869 | 780 | 934 | 975 | 720 |
| E | 179163 | 176349 | 13813 | 13883 | 26043 | 23174 | 70883 | 60278 | 84584 | 84501 | 128287 | 113182 |
| F | 857 | 817 | 917 | 1042 | 972 | 879 | 826 | 787 | 986 | 995 | 830 | 1078 |
| G | 220333 | 197135 | 16044 | 15325 | 29692 | 24542 | 76796 | 60995 | 99625 | 80255 | 133081 | 107111 |
| H | 838 | 996 | 845 | 949 | 968 | 863 | 913 | 1073 | 796 | 981 | 892 | 1066 |
| I | 189530 | 186596 | 16645 | 15886 | 29472 | 26549 | 85791 | 71037 | 105288 | 91399 | 140429 | 114727 |
| J | 732 | 892 | 644 | 836 | 665 | 720 | 935 | 577 | 740 | 718 | 617 | 890 |
| K | 183039 | 163861 | 14628 | 14480 | 28246 | 8286 | 67333 | 8546 | 102877 | 92621 | 126951 | 115636 |
| L | 803 | 754 | 863 | 807 | 802 | 1010 | 792 | 1160 | 625 | 712 | 826 | 699 |
| M | 164407 | 158061 | 15770 | 14717 | 29957 | 25132 | 77606 | 14490 | 99008 | 90770 | 142263 | 122523 |
| N | 717 | 844 | 818 | 785 | 769 | 607 | 637 | 763 | 740 | 816 | 769 | 776 |
| O | 159216 | 153395 | 12612 | 13813 | 27823 | 28453 | 76836 | 76759 | 99142 | 90988 | 137000 | 137816 |
| P | 692 | 731 | 833 | 892 | 871 | 705 | 678 | 797 | 853 | 857 | 630 | 768 |

TABLE 5B
continuation of TABLE 5A.

| | 13 | 14 | 15 | 16 | 17 | 18 | 19 | 20 | 21 | 22 | 23 | 24 |
|---|---|---|---|---|---|---|---|---|---|---|---|---|
| A | 102570 | 94701 | 148880 | 114214 | 129989 | 114058 | 120831 | 101031 | 78131 | 79965 | 1036 | 1072 |
| B | 882 | 974 | 977 | 971 | 945 | 976 | 819 | 825 | 1014 | 986 | 1106 | 876 |
| C | 123863 | 111998 | 151930 | 127488 | 154701 | 141097 | 152062 | 139061 | 70675 | 69277 | 1046 | 1019 |
| D | 885 | 809 | 925 | 1036 | 836 | 911 | 1028 | 874 | 843 | 902 | 896 | 870 |
| E | 134871 | 117315 | 148717 | 145062 | 173918 | 150052 | 176954 | 148696 | 76836 | 75402 | 932 | 842 |
| F | 942 | 845 | 682 | 893 | 721 | 909 | 725 | 787 | 800 | 741 | 889 | 898 |
| G | 135303 | 125264 | 162734 | 157919 | 167199 | 154344 | 163222 | 68627 | 70104 | 66381 | 975 | 917 |
| H | 864 | 1196 | 694 | 946 | 926 | 806 | 963 | 921 | 1124 | 936 | 1051 | 935 |
| I | 129093 | 140694 | 167976 | 152506 | 179449 | 176404 | 170570 | 149261 | 76882 | 64422 | 4573 | 5069 |
| J | 660 | 732 | 896 | 798 | 868 | 868 | 840 | 810 | 719 | 775 | 805 | 701 |
| K | 145268 | 124747 | 157499 | 146890 | 182708 | 163363 | 145072 | 126242 | 77742 | 64328 | 4951 | 5024 |
| L | 617 | 702 | 722 | 617 | 995 | 941 | 754 | 1489 | 769 | 844 | 883 | 848 |
| M | 145181 | 128156 | 158346 | 137852 | 170051 | 161081 | 187963 | 155268 | 71066 | 66637 | 5156 | 4629 |
| N | 756 | 851 | 760 | 809 | 871 | 896 | 865 | 935 | 803 | 760 | 798 | 813 |
| O | 169922 | 111666 | 153677 | 141745 | 174304 | 164863 | 204703 | 174388 | 79720 | 73587 | 5219 | 5999 |
| P | 607 | 767 | 723 | 753 | 689 | 726 | 760 | 563 | 785 | 825 | 822 | 783 |

We claim:

1. A cancer treatment formulation, comprising a dosage form for administration of at least one pro-drug compound for combination with at least one metal ion, wherein the pro-drug compound is selected by in silico screening for association with the selected metal ion and a proteasome, wherein the screening indicates association with the protein only in the presence of the metal ion, and wherein the metal ion is at elevated levels in cancer cells.

2. The cancer treatment formulation according to claim 1, wherein the metal ion is selected from copper, zinc, nickel, and iron.

3. The cancer treatment formulation according to claim 1, wherein the pro-drug compound is selected from: 3,4-dihydroxybenzoic acid; galloflavin; 2-{[(carbamoylsulfanyl)acetyl]amino}benzoic acid; 6,7-Dihydroxycoumaranone; 3,6-bis(hydroxymethyl)pyridazin-4(1H)-one; and 4′,5,7-trihydroxyisoflavone.

4. The cancer treatment formulation according to claim 1, wherein the protein is a proteasome.

5. The cancer treatment formulation according to claim 1, wherein the pro-drug compound is administered orally or intravenously.

6. The cancer treatment formulation according to claim 1, wherein the pro-drug compound is 6,7-Dihydroxycoumaranone at a concentration of 3 μM and the metal ion is copper at a concentration of 100 nM.

7. A computer program, comprising code for determining pro-drug candidates for protein inhibition, comprising in silico modeled of a candidate pro-drug compound with a metal ion and a protein active site, wherein the candidate pro-drug compound complexes with the metal ion to form a metal pro-drug compound complex, wherein a calculation of the metal pro-drug metal complex with the proteasome active site by quantum mechanical methods are carried out for association to an amino acid residue of the protein's active site.

8. The program according to claim 7, wherein the candidate pro-drug compound is selected from the NCI Diversity Set IV and the University of Illinois Marvel Library.

9. The program according to claim 7, wherein the metal ions are copper ions.

10. The program according to claim 7, wherein the protein is a proteasome.

11. The program according to claim 10, wherein the proteasome is the 20S proteasome.

12. A diagnostic or theranostic agent, consisting of a formulation comprising a pro-drug compound that promotes apoptotic turnover by formation of complexes with metal ions that associate with proteasome active centers to induce elevated populations of apoptotic biomarkers in a patient's blood upon delivery of the formulation.
